(19) United States
(12) Patent Application Publication
(10) Pub. No.: US 2023/0140375 A1
(43) Pub. Date: May 4, 2023

(54) METHOD OF DETECTING ERP SIGNAL BASED ON HEART-RATE EVOKED POTENTIAL

(71) Applicant: KOREA INSTITUTE OF SCIENCE AND TECHNOLOGY, Seoul (KR)

(72) Inventors: Sang In PARK, Seoul (KR); Ji Hyeon HA, Seoul (KR); Laehyun KIM, Seoul (KR)

(73) Assignee: KOREA INSTITUTE OF SCIENCE AND TECHNOLOGY, Seoul (KR)

(21) Appl. No.: 17/874,563

(22) Filed: Jul. 27, 2022

(30) Foreign Application Priority Data
Oct. 28, 2021 (KR) 10-2021-0145866

(51) Int. Cl. A61B 5/00; A61B 5/352; A61B 5/377

(52) U.S. Cl. CPC A61B 5/7282; A61B 5/352; A61B 5/377; A61B 5/7217

(57) ABSTRACT

Provided are a method and apparatus for detecting an event-related potential (ERP), the method including: detecting an R-peak signal by detecting an electrocardiogram (ECG) signal of a subject via an ECG sensor; inducing an evoked potential to the subject by presenting an ERP stimulus to the subject at a certain period on basis of the R-peak signal; and detecting an electroencephalogram (EEG) signal of the subject exposed to the ERP stimulus by using an EEG sensor, and extracting an ERP signal from the EEG signal, wherein intermixture of a heart-rate evoked potential (HEP) of the subject with the ERP signal is inhibited by removing the ERP stimulus being presented to the subject during the certain period, after a latency by a certain time from a point in time the R-peak occurs.

### CROSS-REFERENCE TO RELATED APPLICATION

This application is based on and claims priority under 35 U.S.C. § 119 to Korean Patent Application No. 10-2021-0145866, filed on Oct. 28, 2021, in the Korean Intellectual Property Office, the disclosure of which is incorporated by reference herein in its entirety.

### BACKGROUND

### 1. Field

One or more embodiments relate to a method and apparatus for detecting an event-related potential (ERP) signal, and more particularly, to a method and apparatus for detecting an ERP signal having noise reduced by an electrocardiogram (ECG).

### 2. Description of the Related Art

An event-related potential (ERP) may refer to a potential difference appearing in the brain in response to an event such as any sensory or cognitive stimulus or movement, and may be analyzed via an electroencephalogram (EEG) detected in a non-invasive method.

In ERP analysis based on an EEG, various types of noise signals lower the accuracy of the ERP analysis. Sources of contamination of such ERP signals appear in various forms. Examples of such contamination include contamination due to a change in a sensor contact by movement of a subject wearing an EEG measurement sensor, signal contamination by an effect of an electromyogram (EMG), signal contamination by a wearer's blinking, signal contamination by an electrooculogram (EOG), signal contamination by an electrocardiogram (ECG), and the like.

Existing ERP research has made efforts to control a subject during an experiment as well as to minimize the subject's movement while presenting a stimulus to reduce signal contamination as described above.

A heart-rate evoked potential (HEP) is an indicator that reflects brain-heart connectivity, and is synchronized with an alpha rhythm of an EEG on the basis of an R-peak in a QRS waveform of an ECG appearing by the heartbeat. The HEP is divided into a first component and a second component.

The first component of the HEP is an evoked potential as an indicator reflecting a rate at which the heart's neurological information reaches the cerebrum from the vagus nerve of the heart through the afferent nerve pathway, and the second component of the HEP is an evoked potential as an indicator reflecting a rate at which the heart's blood pressure wave reaches the cerebrum from the vagus nerve of the heart through the afferent nerve pathway.

The HEP is transferred to the cerebrum, and acts as a main noise signal that contaminates an ERP signal, thereby lowering the accuracy of ERP analysis. In spite of the above issue, existing ERP analysis does not consider the effect of the HEP. Non-consideration of the effect of the HEP is because, during an experiment, a subject's movement, blinking of eyes, and the like may be controlled via a noise removal algorithm, but an EEG signal with an HEP induced by an ECG may not be controlled.

Therefore, for an accurate analysis of an ERP, there is a need for studies on an ERP analysis method capable of excluding an HEP from an ERP.

### SUMMARY

One or more embodiments include a method and apparatus for detecting an event-related potential (ERP) signal, capable of reducing noise.

One or more embodiments include a method and apparatus for detecting an ERP signal, capable of increasing the accuracy of ERP analysis by effectively excluding an effect of a heart-rate evoked potential (HEP).

Additional aspects will be set forth in part in the description which follows and, in part, will be apparent from the description, or may be learned by practice of the presented embodiments of the disclosure.

According to one or more embodiments, a method of detecting an ERP signal includes: detecting an R-peak signal by detecting an electrocardiogram (ECG) signal of a subject by an ECG sensor; inducing an evoked potential to the subject by presenting an ERP stimulus to the subject for a certain period on the basis of the R-peak signal; and detecting, via an electroencephalogram (EEG) sensor, an EEG signal of the subject exposed to the ERP stimulus and extracting an ERP signal from the EEG signal via a signal processing unit, wherein, after a latency by a certain time from a point in time when the R-peak occurs, the ERP stimulus presented to the subject during the certain period is removed to inhibit intermixture of an HEP of the subject with the ERP signal.

According to one or more embodiments, a method of detecting an ERP signal includes: detecting an R-peak signal by detecting an electrocardiogram (ECG) signal of a subject by an ECG sensor; inducing an evoked potential to the subject by presenting ERP stimulus to the subject at a certain period on the basis of the R-peak signal; and detecting an EEG signal of the subject exposed to the ERP stimulus by using an EEG sensor and extracting an ERP signal from the EEG signal, wherein, after a latency by a certain time from a point in time when the R-peak occurs, the ERP stimulus being presented to the subject during the certain period is removed to inhibit intermixture of an HEP of the subject with the ERP signal.

According to one or more embodiments, the certain period may include a time range corresponding to at least one of a first component and a second component of the HEP.

According to one or more embodiments, the ERP stimulus may be presented to the subject after about 50 ms to about 600 ms from the point in time when the R-peak occurs.

According to one or more embodiments, the HEP may include a first component and a second component, and the ERP stimulus may be presented to the subject from a time point beyond a point in time when the first component of the HEP occurs.

According to one or more embodiments, the HEP may be detected from the subject, and the certain period for which the ERP stimulus is not presented may be calculated from the HEP obtained from the subject.

According to one or more embodiments, an apparatus for detecting an ERP signal includes: a simulator configured to induce an evoked potential to a subject by presenting an ERP stimulus to the subject; an ECG measurer having an ECG sensor configured to detect an ECG signal from the subject exposed to the ERP stimulus; an EEG measurer having an EEG sensor configured to detect an EEG signal from the subject; a signal processing unit configured to detect an HEP signal by detecting an R-peak from the ECG signal, and detect an ERP signal from the EEG signal; and an ERP stimulus controller configured to inhibit intermixture of an HEP of the subject with the ERP signal by removing the ERP stimulus presented to the subject during a certain period, after a latency by a certain time from a time point of occurrence of the R-peak detected from the ECG signal by the signal processing unit.

According to one or more embodiments, the certain period may include a time range corresponding to at least one of a first component and a second component of the HEP.

According to one or more embodiments, the ERP stimulus may be presented to the subject after about 50 ms to about 600 ms from the point in time when the R-peak occurs.

According to one or more embodiments, the HEP may include a first component and a second component, and the ERP stimulus may be presented to the subject from a time point beyond a point in time when the first component of the HEP occurs.

According to one or more embodiments, the HEP may be detected from the subject, and the certain period for which the ERP stimulus is not presented may be calculated from the HEP obtained from the subject.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A and 1B illustrate a stimulus presentation monitor according to one or more embodiments;

FIG. 2 illustrates a subjective scale for a mental workload (MWL) used in an experiment, according to an embodiment;

FIG. 3A illustrates photos of a system applied to an experiment according to an embodiment; FIG. 3B is a block diagram illustrating an experimental process according to an embodiment;

FIG. 4A illustrates an electroencephalogram (EEG) signal and an electrocardiogram (ECG) signal, according to an embodiment; FIG. 4B are graphs illustrating the results of an event-related potential (ERP) signal according to whether or not an ECG is considered;

FIG. 5A illustrates accuracy with respect to a target in an experiment according to one or more embodiments; FIG. 5B illustrates a response time with respect to a target in an ERP task; FIG. 5C illustrates the result of a paired sample T-test for an SMEQ between low-MWL and high-MWL conditions;

FIGS. 6A to 6C are graphs illustrating the results of statistical analysis for respective EEG channels in an experiment, according to one or more embodiments;

FIGS. 7A to 7C are statistical graphs illustrating, for respective channels, characteristics at 600 ms with respect to a time value (latency) of a peak in an experiment, according to one or more embodiments;

FIGS. 8A to 8E are graphs illustrating scatter plots with respect to channels F3, F4, P4, O1, and O2 from among EEG channels in an experiment, according to one or more embodiments, i.e., illustrate scatter plots of a high-MWL and a low-MWL with respect to ERP_{T}, ERP_{HEP}, and ERP_{A-HEP} conditions floated on orthogonal coordinates including latency (an x axis) and amplitude (a y axis);

FIG. 9 illustrates receiver operation characteristic (ROC) curves for ERP_{T}, ERP_{HEP}, and ERP_{A-HEP} according to an RBF-SVM classifier in an experiment, according to one or more embodiments; and

FIG. 10 illustrates an internal algorithm of an apparatus for detecting the entire ERP, according to one or more embodiments.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, example embodiments will be described in detail with reference to the accompanying drawings. The embodiments may, however, be modified in various other forms, and the scope of the present disclosure should not be construed as being limited by the embodiments described in detail below. The embodiments of the present disclosure are provided so that the disclosure will be thorough and complete, and will fully convey the concept of the disclosure to one of ordinary skill in the art. Like reference numerals in the drawings denote like elements. Furthermore, various elements and regions in the drawings are schematically drawn. Therefore, the present disclosure is not limited by a relative size or interval drawn in the accompanying drawings.

Although the terms “first,” “second,” etc. may be used herein to describe various elements, these elements should not be limited by these terms. These terms are only used to distinguish one element from another element. For example, a first element could be termed a second element and, similarly, a second element could be termed a first element, without departing from the scope of example embodiments.

The terminology used herein is for the purpose of describing particular embodiments only and is not intended to be limiting of example embodiments. The singular forms are intended to include the plural forms as well, unless the context clearly indicates otherwise. It will be further understood that the terms “comprise,” “comprising,” “include,” “including,” “have,” and/or “having” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which example embodiments belong. Also, it will be further understood that terms, such as those defined in commonly used dictionaries, should be interpreted as having a meaning that is consistent with their meaning in the context of the relevant art and will not be interpreted in an idealized or overly formal sense unless expressly so defined herein.

In cases where certain embodiments may be implemented differently, a particular process order may be performed differently from the described order. For example, two processors described in succession may be performed substantially simultaneously, or may be performed in an order opposite to the described order.

In one or more embodiments, a sensing apparatus for detecting, from a human body, an event-related potential (ERP) signal in which an effect of a heart-rate evoked potential (HEP) is minimized, or a stimulus display apparatus or stimulus presentation apparatus, which induces the generation of an ERP signal by causing a mental workload (MWL), may apply a computer system including a monitor. The main body of the computer system includes hardware having a central processing unit, a peripheral controller, a memory, a storage, and the like, and an analysis apparatus in the form of software that is stored in the memory or storage and activated by the central processing unit to perform various types of analysis described below.

In the embodiment, in an HEP signal recognized as an ERP, a first component, which reflects a rate at which neurological information of the heart reaches the cerebrum, occurs at about 50 ms to about 250 ms on the basis of an R-peak of an electrocardiogram (ECG). Also, a second component, which reflects a rate at which a blood pressure wave of the heart reaches the cerebrum, occurs at about 250 ms to about 600 ms on the basis of the R-peak of the ECG. The periods of the occurrence of the first component and the second component of the HEP are slightly different for each individual, and thus, individual HEP characteristics need to be secured via an ECG for each individual.

In the embodiment, an ERP signal is detected while controlling presentation of ERP stimulus to a subject so that an HEP signal affecting the cerebrum as described above is not included as noise in the ERP signal. Also, the ERP signal obtained as described above is compared with an ERP signal detected by an existing method to analyze a difference therebetween.

Hereinafter, a several-stage experiment, a verification process, and the like performed to analyze an ERP signal by considering an HEP will be described in phase.

1. Subjects

14 university students (7 males, 7 females, average age: 25.2±3.4) participate in an experiment. All the subjects have no abnormalities or medical history in cardiovascular nervous systems and central nervous systems, and are asked to take sufficient sleep the day before. In addition, intake of caffeine, smoking, alcohol, and the like is prohibited the day before the experiment. The experiment is conducted after explaining, before the experiment, approximate matters of the experiment except for the aim of research to all the subjects participating in the experiment, and also a certain amount of money is paid in return for the experiment.

2. Experimental Stimulus

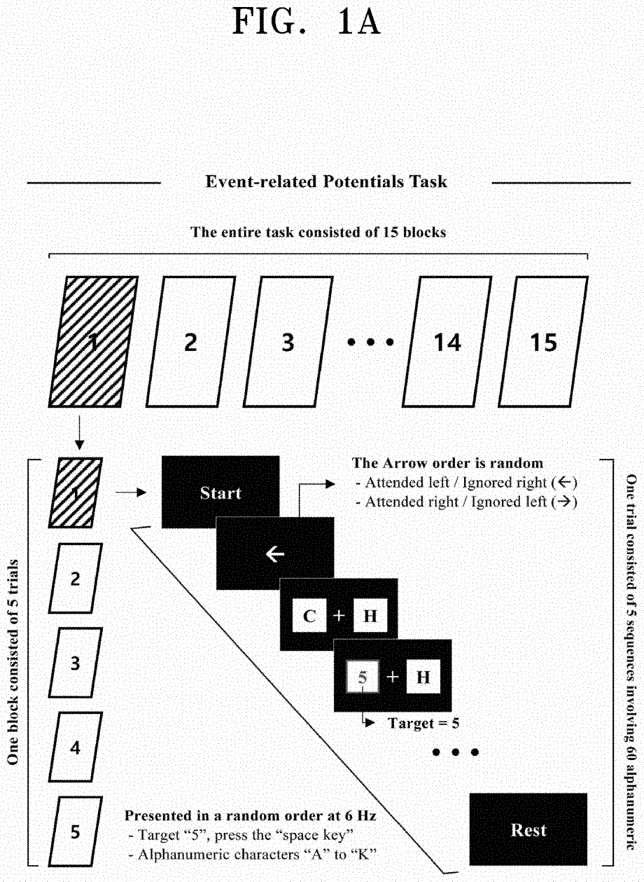

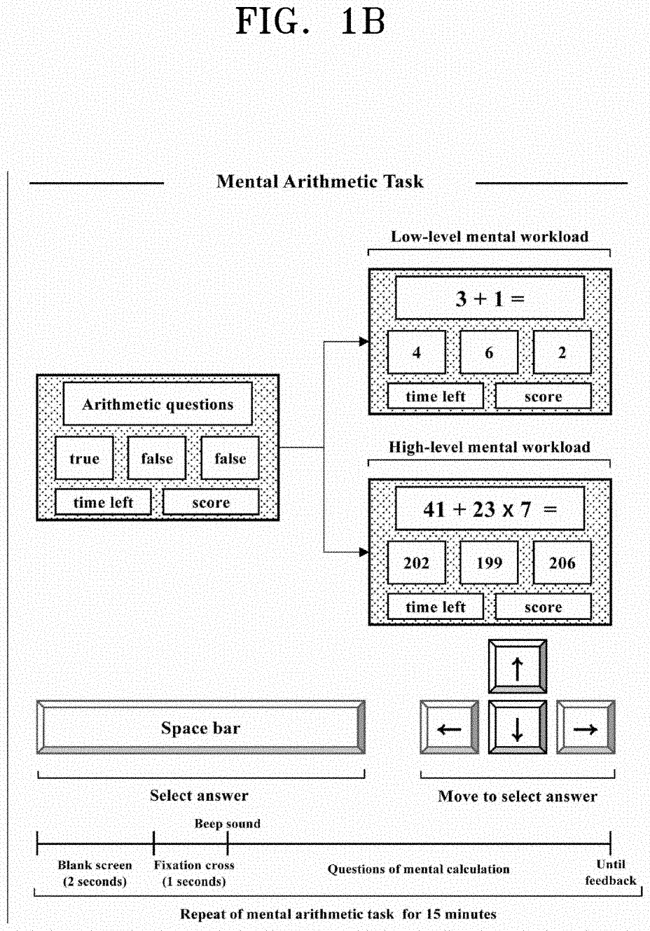

A mental arithmetic task is performed to observe an ERP response according to a mental workload (MWL). Single-digit addition and subtraction are performed to give a low-MWL. Mental arithmetic, which includes addition, subtraction, multiplication, and division with double digits, is performed to give a high-MWL (15 minutes). All the subjects perform an ERP experiment before and after mental arithmetic tasks as described above (15 minutes). FIGS. 1A and 1B respectively illustrate ERP task stimulus and mental arithmetic task stimulus displayed to the left and right of a single monitor screen, i.e., FIG. 1A illustrates ERP task stimulus used in an experiment, and FIG. 1B illustrates mental arithmetic task stimulus used in the experiment. In the ERP experiment, a subject looks at a “+” (fixation) screen, and then presses a space bar on a keyboard when “5” appears.

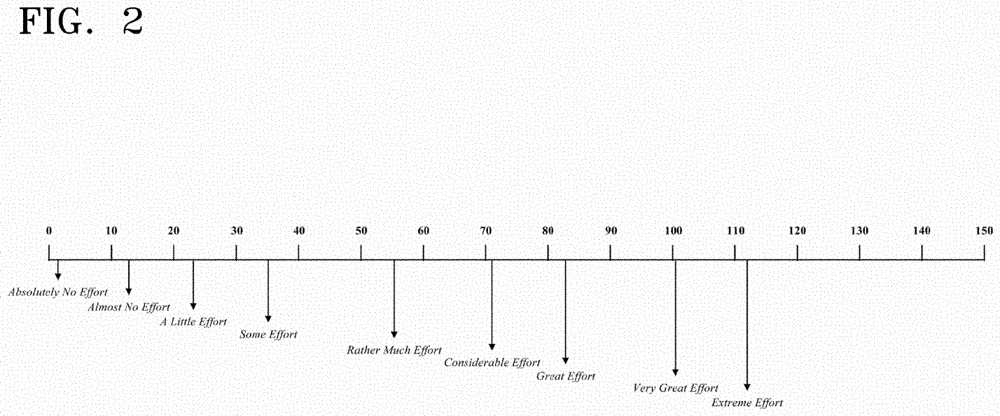

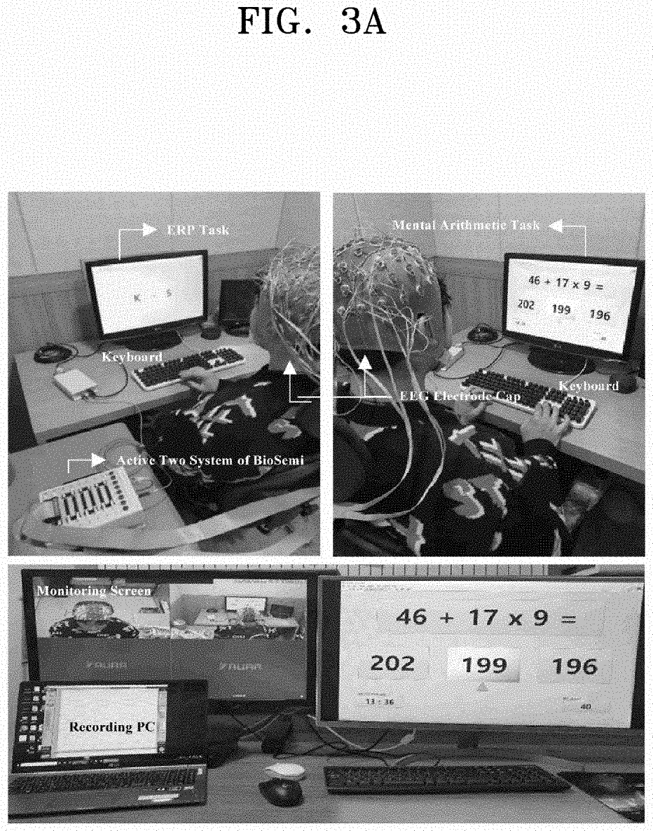

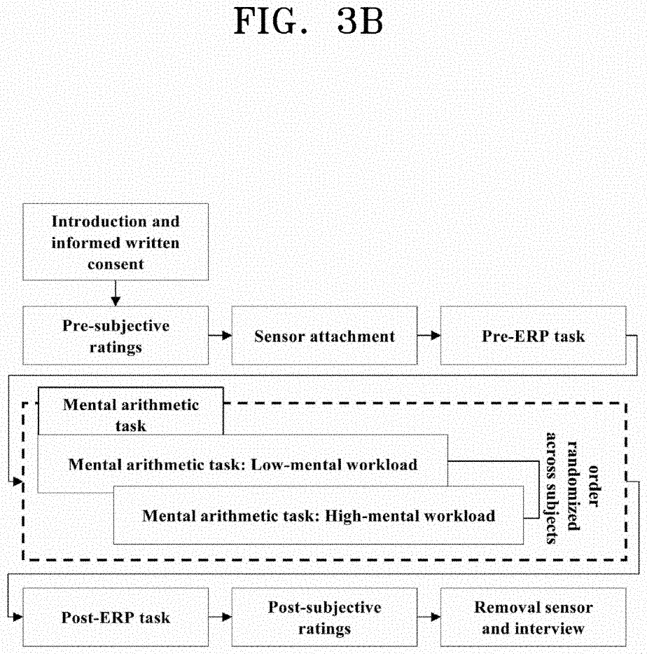

FIG. 2 illustrates a subjective scale for an MWL used in an experiment, which follows a scale of a subjective mental effort questionnaire (SMEQ). FIG. 3A illustrates photos of a system applied in the present experiment, and FIG. 3B is a block diagram illustrating an experimental process.

3. Experimental Method

As illustrated in FIG. 3A, the photo at the left top shows an ERP task screen (an ERP simulator), and the photo at the right top shows a mental arithmetic task screen (a mental arithmetic simulator). Also, the photo at the bottom in FIG. 3A shows a monitoring screen for a researcher.

As illustrated in FIG. 3A, a subject, who participates in an experiment, seats in a comfortable chair and is presented with stimulus via a monitor (in the present experiment, a 24″ LED monitor). The subject conducts the experiment while being 60 cm away from the monitor. An electroencephalogram (EEG) and an ECG are measured when conducting the experiment (Active-two, Biosemi S. V., Amsterdam, Netherlands). An ERP experiment of 15 minutes (FIG. 1A before a mental arithmetic task, a mental arithmetic task of 15 minutes (FIG. 1B), and an ERP experiment of 15 minutes (FIG. 1A) after the mental arithmetic task are performed.

All the subjects are divided into two days and randomly perform a low-MWL experiment and a high-MWL experiment, respectively. An experimental reward amount of 150% is promised to be paid to subjects with scores of the top 15% of an MWL experiment, to increase the willingness of the subjects to participate in the MWL experiment.

In an MWL experiment (an ERP task, a mental arithmetic task) as illustrated at the right top in FIG. 1A, an EEG, which appears when a particular target, “5” in the present embodiment, appears as a target, is used.

The ERP simulator and the mental arithmetic simulator are located on the left and right sides of a screen on one monitor, and the subjects are asked to focus attention on the indicated side of the screen according to an arrow. (Ignore the left, pay attention to the right, vice versa). A simulator includes 12 alphanumeric representations including non-targets (“A” to “K”) and a target (“5”). Alphanumeric characters are randomly updated at a rate of 6 Hz. One trial includes five sequences involving 60 alphanumeric characters lasting during 10 seconds at a trial interval of 2 seconds (60 seconds). One block includes five trials, and the entire task includes 15 blocks. The targets are presented with a probability of 5% within a one-time trial, and an interval between the targets lasts less than 1 second to avoid ERP duplication during analysis.

As illustrated in FIG. 1A, a left or right arrow appears, and the subjects are asked to focus on the direction of the appearing arrow. Here, the subjects repeatedly perform a process of pressing the space bar on the keyboard when the target “5” appears. EEGs appearing at this time are collected and analyzed, and the results of EEGs, which appear differently after performing some difficult arithmetic and then performing easy arithmetic, according to an MWL experiment such as mental arithmetic illustrated in FIG. 1B, are used.

In detail, a mental arithmetic task is designed to induce an MWL on the basis of previous studies (So et al., 2017; Jost et al., 2019). The mental arithmetic task is divided into two task levels that are a low-MWL task and a high-MWL task. The low-MWL task includes easy questions related to single-digit addition and subtraction (i.e., 3+2, 4-1, the range of 1 to 9). The high-MWL task includes difficult questions related to mixed arithmetic operations (i.e., 36×7−24, 43+72/9, a range of 1 to 99). Mental arithmetic questions are presented randomly within a defined range, and include the results of one correct answer and two incorrect answers. The two incorrect answers are automatically calculated by randomly adding to or subtracting from a correct answer in the range of 1 to 5. The subjects need to select the correct answer by using arrows and the space bar on the keyboard as illustrated in FIG. 1B. The ERP and mental arithmetic tasks have been developed by using LabVIEW2016 (National Instruments Inc., Austin, Tex., USA).

In the experiment described above, the subjects are asked to report MWL statuses as subjective ratings before and after the experiment. An SMEQ (Sauro and Dumas, 2009), which is a questionnaire on a scale of 0 to 150 for rating an amount of MWL, is used. The subjects perform a pre-ERP task for 15 minutes. While this session progresses, all the subjects are asked to fix gazes on a red cross at the center of the screen that is 60 cm away from a display and press the space bar when the target “5” is presented. Accuracy and a response time with respect to the target are measured.

A mental arithmetic task is performed for 15 minutes subsequent to the pre-ERP task, and all the subjects are asked to select the correct answer to the mental arithmetic questions from among three options by using the arrow keys and the space bar on the keyboard. Each of the subjects receives 10 scores for the correct answer and receives a deduction of 10 scores for the incorrect answer. 150% of the experimental reward amount is paid to the subjects who receive scores of the top 15% to increase the motivation and immersion of the subjects. The subjects are divided into low-MWL and high-MWL task groups. A low-MWL or high-MWL task is performed on the first day, and another MWL task is performed on the next day (e.g., a low-MWL task on the first day, a high-MWL task on the second day, an order randomized across subjects). The subjects then perform the same post-ERP task as the pre-ERP task. The experimental environment and procedure are as illustrated in FIGS. 3A and 3B.

4. Extraction and Analysis of Signal

An EEG signal is recorded at a sampling rate of 2,048 Hz in 64 channels mounted on an EEG electrode cap (Active-two, Biosemi SV, Amsterdam, Netherlands) based on the international 10-20 montage with a separate reference electrode and ground electrode for each system. (Common mode detection, CMS and drive right leg, DRL). Impedance of all electrodes is maintained less than 5 kΩ and less than 10 kΩ for two eye channels. The measured EEG signal is down-sampled to 512 Hz, and a common average reference (CAR) procedure is used (Perrin et al., 1989). A CAR is calculated by subtracting each channel from an average potential for all channels.

Preprocessing is minimized by specifying a threshold value for each trial to prevent significant ERP patterns from being contaminated. In an experiment in which an amplitude exceeds±100 μV in all electrodes, a messy EEG, which is higher than or equal to 100 μV, is removed by performing independent component analysis (ICA). The EEG signal is cut to a length of 1000 ms from −200 ms before a start of stimulus to 800 ms after the start of the stimulus. EEG signals in all tasks are obtained by subtracting an average value of signals from about −200 ms to about 0 ms and averaging the signals to obtain an average ERP signal. Here, a maximum value of the average ERP signal within about 530 ms to about 750 ms and a time value at that time are acquired and used as characteristics of a P600 component to extract P600 characteristics that show a difference according to an MWL. EEG channels used in the analysis of the present experiment are channels F3, F4, C3, C4, P3, P4, O1, and O2. All signal processing and data analysis are performed by using MATLAB toolbox EEGlab (2020b, Mathworks Inc., Natick, Mass., USA).

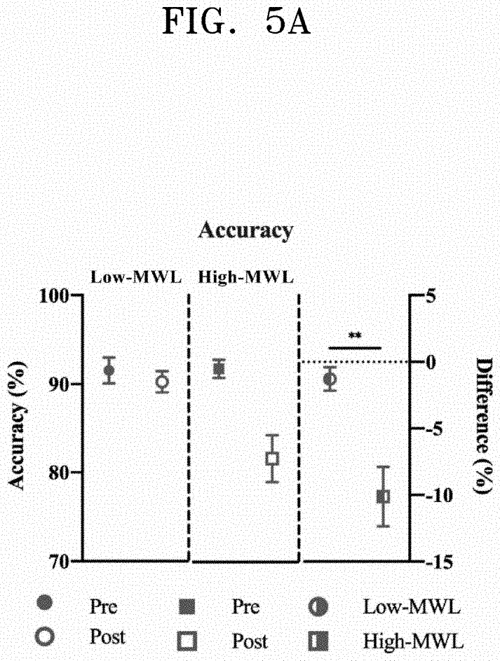

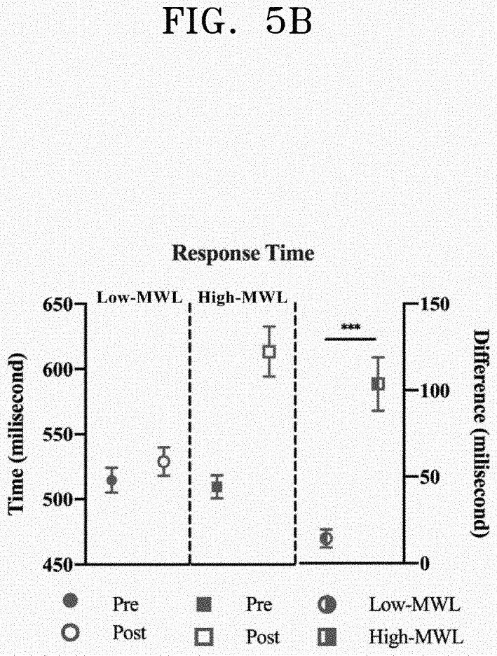

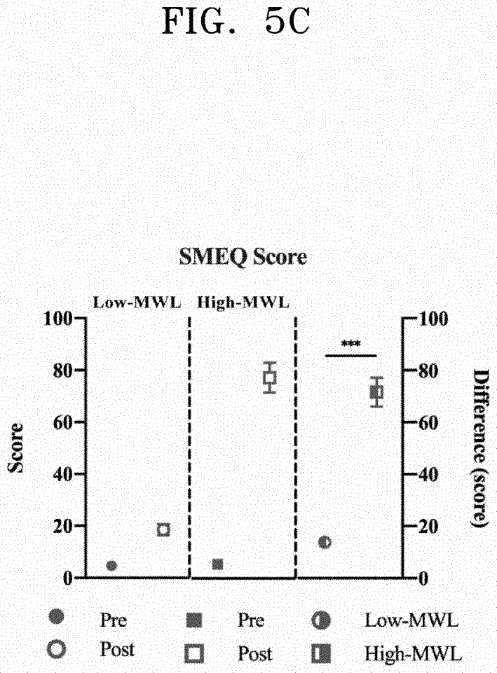

For a subject's ECG, an R-peak is extracted via a QRS detection algorithm, and a time point of the R-peak is extracted. Accordingly, ERP signals of about 0 ms to about 800 ms are divided into ERP signals that are affected by an HEP and ERP signals that are not affected by the HEP. An ERP (hereinafter, ERP_{A-HEP}) signal, which is not affected by an HEP, needs to have no R-peak within about 280 ms to about 700 ms, so that a response of about 50 ms to about 250 ms, which is a first component of the HEP, is not included in about 530 ms to about 750 ms including a P600 component. In contrast, an ERP (hereinafter, ERP_{HEP}) signal affected by the HEP needs to have an R-peak within about 280 ms to about 700 ms. The ERP signals divided as described above are used as characteristics for measuring an MWL. FIGS. 5A and 5B illustrate examples of signal processing among ERP_{A-HEP}, ERP_{HEP}, and ERP_{T}.

5. Result of Experiment

A P600 amplitude of each subject's pre-/post-ERP signal and a latency time value at that time are divided into a low-MWL and a high-MWL and compared. Statistical analysis is performed on the low-MWL and the high-MWL by using differences in the amplitude and time value of the pre-/post-ERP signal as characteristics of each subject. A statistical technique uses a paired sample T-test. A statistical analysis program uses MATLAB 2020b.

For the analysis described above, statistical analysis, and classification analysis using machine learning are performed according to a condition using the total ERP signal (hereinafter, ERP_{T}), an ERP_{A-HEP} condition, and an ERP_{HEP} condition to observe a difference between the ERP_{A-HEP} and ERP_{HEP} conditions. Statistical significance is statistically significant only when a statistical value P is lower than 0.0031 by using Bonferroni correction which corrects as much as comparatively analyzed numbers.

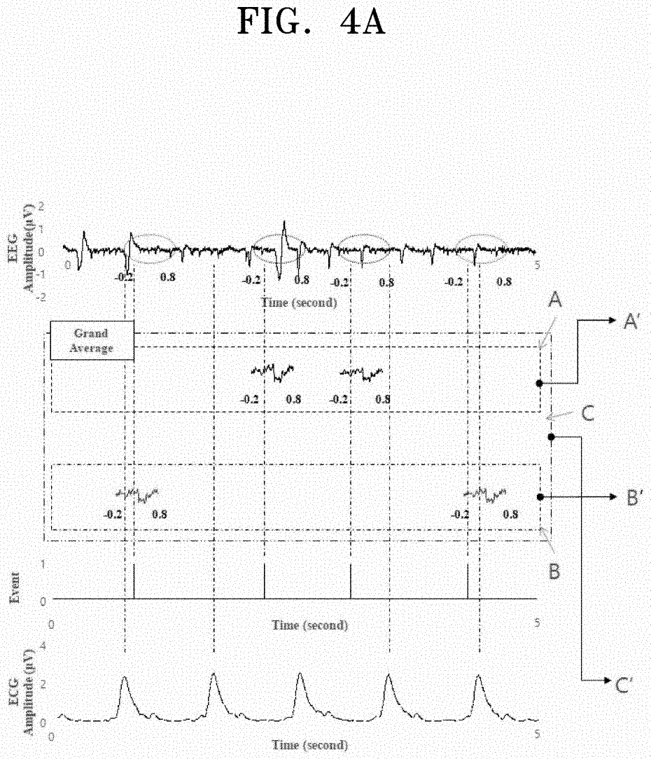

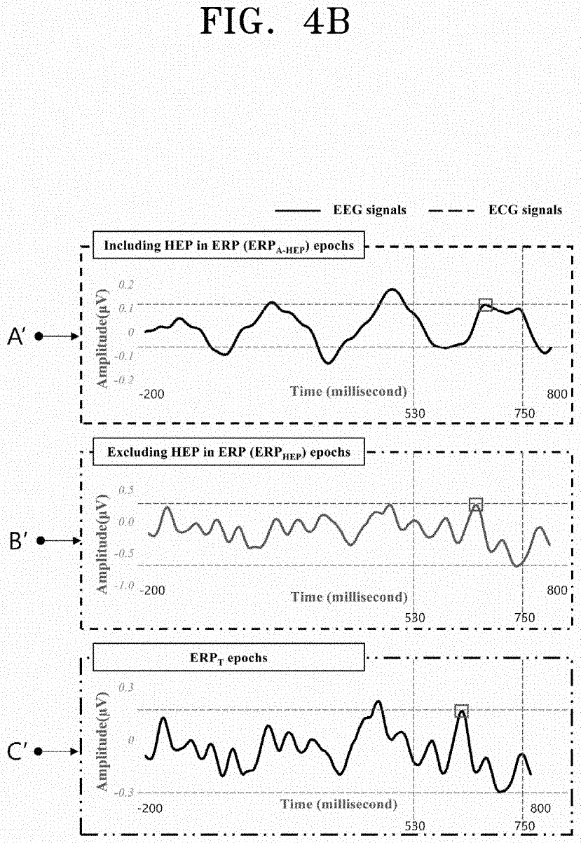

FIG. 4A illustrates an EEG signal and an ECG signal. FIG. 4B illustrates graphs showing the results of an ERP signal according to whether or not an ECG is considered.

FIGS. 4A and 4B illustrate signal processing with respect to ERP_{A-HEP} (A, A′) for which an ECG is not considered, ERP_{HEP} (B, B′) for which an ECG is considered, and mixed ERP_{T} (C, C′) for which an ECG is not considered.

An EEG signal having a fine waved pattern at the top of FIG. 4A is affected by a peak of an ECG signal at the bottom of FIG. 4A, and accordingly, an EEG measured at this time is acquired by a grand average.

When only an EEG (A) for which an ECG is considered is collected, characteristics at about 600 ms in a section of about 530 ms to about 750 ms appear well as shown at the right top A′. Also, when an ECG is not considered as shown at a portion C, as shown at a portion B′ of FIG. 4B, the characteristics at about 600 ms in the section of about 530 ms to about 750 ms do not appear well.

As a result, when the ECG is not considered, a result in the section of about 530 ms to about 750 ms as shown at a portion C′ of FIG. 4B is obtained. Accordingly, a peak may be sufficiently found, but sometimes, an inaccurate ERP may be obtained.

6. Result of Statistical Analysis

-Subjective Rating-

FIGS. 5A to 5C illustrate the results of statistical analysis of a subjective rating for a subject before analysis of an EEG.

In detail, FIG. 5A illustrates accuracy with respect to a target, FIG. 5B illustrates a response time with respect to a target in an ERP task, and FIG. 5C illustrates the result of a paired sample T-test for an SMEQ between low-MWL and high-MWL conditions (**p>0.0031, ***p>0.001).

As illustrated in FIG. 5A, the accuracy is not substantially significantly different at the low-MWL before and after an experiment, and the result is the significant point. A subject is not particularly burdened by a mental arithmetic task, and thus does not show a significant difference in the accuracy and response time of pressing “5” before and after the experiment. However, at the high-MWL, after the experiment, the accuracy relatively decreases, and the response time also increases.

-Objective Rating-

Statistics are analyzed according to each condition according to whether or not an HEP is affected. Tables below (Tables 1 and 2) show statistical results of amplitude and time components of a P600 component in each channel of an EEG at the high-MWL and the low-MWL.

No statistically significant channel is present in an ERP_{HEP} condition, and the channel O1 is statistically significant only at a P600 time value in the ERP_{T} condition. In contrast, in the ERP_{A-HEP} condition, channels F3, F4, P4, and O1 are significant at the amplitude, and channels F3, F4, P4, O1, and O2 are significant at the time value.

Table 1 below shows the results of statistical analysis of a P600 amplitude in respective conditions, and Table 2 below shows the results of statistical analysis of a P600 time value in respective conditions.

TABLE 1

| | | | | | | | | EffectSize | |
|---|---|---|---|---|---|---|---|---|---|
| | Site | Condition | N | Mean | SD | t | p | Hedges’ g | 95% CI |
| ERP [symbol] | F [symbol] | Low-MWL | 14 | 0.03 | 0.09 | 2.863 | .0133 | −1.046 | −1.836~−0.256 |
| | | High-MWL | 14 | −0.05 | 0.09 | | (>.05) | | |
| | P [symbol] | Low-MWL | 14 | 0.08 | 0.09 | 2.784 | .0155 | −1.149 | −1.948~−0.349 |
| | | High-MWL | 14 | −0.03 | 0.04 | | (>.05) | | |
| | O [symbol] | Low-MWL | 14 | 0.04 | 0.09 | 2.556 | 0.239 | −1.043 | −1.833~−0.254 |
| | | High-MWL | 14 | −0.03 | 0.03 | | (>.05) | | |
| | O [symbol] | Low-MWL | 14 | 0.07 | 0.10 | 3.453 | .0043 | −1.444 | −2.276~−0.633 |
| | | High-MWL | 14 | −0.04 | 0.04 | | (<.05) | | |
| ERP [symbol] | O [symbol] | Low-MWL | 14 | 0.07 | 0.10 | 2.658 | .0197 | −1.012 | −1.799~−0.225 |
| | | High-MWL | 14 | −0.01 | 0.05 | | (<.05) | | |
| ERP [symbol] | F [symbol] | Low-MWL | 14 | 0.06 | 0.05 | 5.505 | .0001 | −1.988 | −2.893~−1.082 |
| | | High-MWL | 14 | −0.03 | 0.04 | | (<.001) | | |
| | F [symbol] | Low-MWL | 14 | 0.04 | 0.03 | 4.787 | .004 | −1.265 | −2.075~−0.453 |
| | | High-MWL | 14 | −0.02 | 0.06 | | (>.001) | | |
| | P [symbol] | Low-MWL | 14 | 0.04 | 0.05 | 4.383 | .0007 | −1.449 | −2.281~−0.616 |
| | | High-MWL | | −0.04 | 0.06 | | (>.001) | | |
| | O [symbol] | Low-MWL | 14 | 0.07 | 0.10 | 3.815 | .0021 | −1.391 | −2.217~−0.566 |
| | | High-MWL | 14 | −0.04 | 0.05 | | (>.001) | | |
| | O [symbol] | Low-MWL | 14 | 0.07 | 0.08 | 3.298 | .0058 | −1.499 | −2.337~−0.661 |
| | | | | | | | (>.05) | | |

[symbol]

indicates data missing or illegible when filed

TABLE 2

| | | | | | | | | Effect Size | |
|---|---|---|---|---|---|---|---|---|---|
| | Site | Condition | N | Mean | SD | t | p | Hedges’ g | 95% CI |
| ERP [symbol] | F [symbol] | Low-MWL | 14 | −44.00 | 72.29 | −3.023 | .0098 | 1.208 | 0.403~2.014 |
| | | High-MWL | 14 | 45.00 | 75.02 | | (>.05) | | |
| | P [symbol] | Low-MWL | 14 | −20.29 | 39.21 | −3.284 | .0059 | 1.493 | 0.655~2.331 |
| | | High-MWL | 14 | 39.00 | 40.20 | | (>.05) | | |
| | O [symbol] | Low-MWL | 14 | −8.57 | 18.89 | −3.935 | .0017 | 1.462 | 0.628~2.296 |
| | | High-MWL | 14 | 45.43 | 48.71 | −3.067 | (>.0031) | 0.750 | |
| | O [symbol] | Low-MWL | 14 | −6.86 | 23.60 | −3.067 | 0.0090 | 0.750 | −0.016~1.516 |
| | | High-MWL | 14 | 38.29 | 42.81 | | (<.05) | | |
| ERP [symbol] | F [symbol] | Low-MWL | 14 | −8.43 | 21.95 | −2.425 | .0306 | 1.098 | 0.303~1.893 |
| | | High-MWL | 14 | 39.57 | 57.79 | | (>.05) | | |
| | P [symbol] | Low-MWL | 14 | −4.00 | 30.09 | −3.500 | .0039 | 0.903 | 0.125~1.680 |
| | | High-MWL | 14 | 28.43 | 40.93 | | (>.05) | | |
| | O [symbol] | Low-MWL | 14 | −1.14 | 31.95 | −2.423 | .0308 | 0.830 | 0.058~1.602 |
| | | High-MWL | 14 | 35.00 | 52.62 | | (>.05) | | |
| ERP [symbol] | F [symbol] | Low-MWL | 14 | −24.43 | 35.54 | −4.348 | .0008 | 1.823 | 0.942~2.704 |
| | | High-MWL | 14 | 42.86 | 38.24 | | (>.001) | | |
| | F [symbol] | Low-MWL | 14 | −13.86 | 26.57 | −3.833 | .0021 | 1.533 | 0.690~2.375 |
| | | High-MWL | 14 | 36.14 | 37.71 | | (>.001) | | |
| | P [symbol] | Low-MWL | 14 | −11.86 | 33.92 | −4.283 | .0009 | 1.662 | 0.803~2.521 |
| | | High-MWL | 14 | 35.14 | 21.18 | | (>.001) | | |
| | O [symbol] | Low-MWL | 14 | −7.71 | 14.42 | −5.115 | .0002 | 1.714 | 0.848~2.581 |
| | | High-MWL | 14 | 27.57 | 25.28 | | (>.001) | | |
| | O [symbol] | Low-MWL | 14 | −3.14 | 19.25 | −5.526 | .0001 | 1.871 | 0.983~2.760 |
| | | | | | | | (>.001) | | |

[symbol]

indicates data missing or illegible when filed

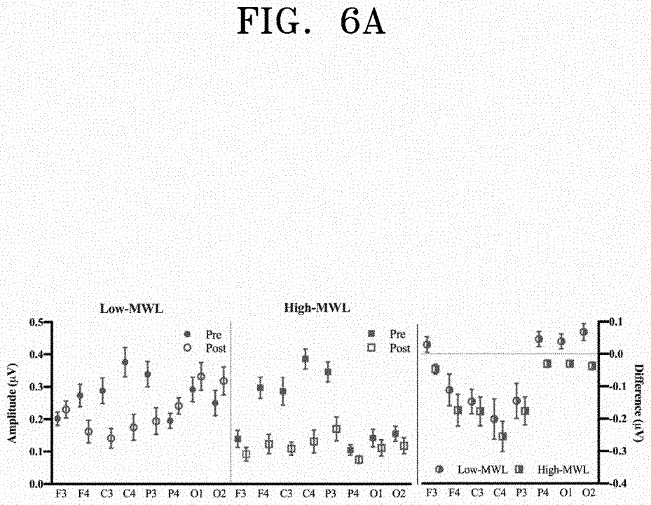

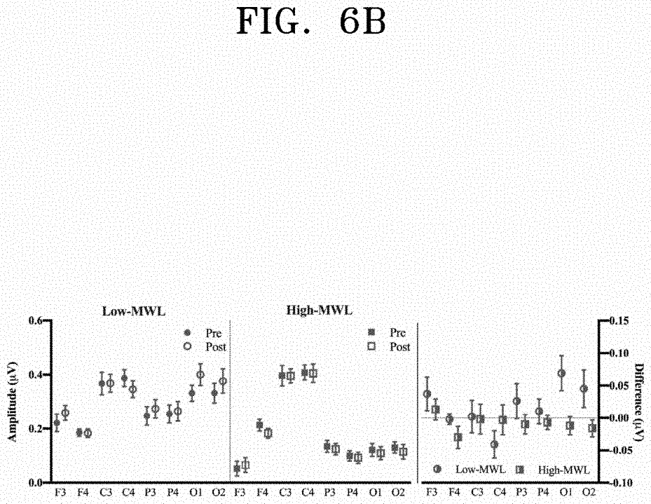

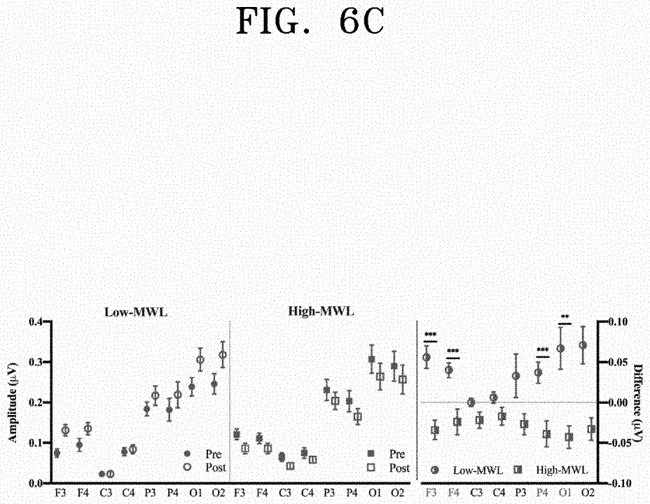

FIGS. 6A to 6C are graphs illustrating the results of statistical analysis for respective channels of an EEG.

FIG. 6A illustrates the result of statistical analysis that does not consider an ECG. FIG. 6B illustrates the result of statistical analysis in which an ECG is considered and included. FIG. 6C illustrates the result of statistical analysis in which an ECG is considered but is not included.

Referring to FIGS. 6A to 6C, when differences in a low-MWL before and after an experiment are compared with differences in a high-MWL before and after the experiment in all channels via statistics, channels showing the most significant differences appear and show how clearly characteristics of an EEG appear. These clear differences are shown well in FIG. 6C. ***(p<0.001) is most statistically significant. Here, the significance of*indicates a result that is considered not to be significant in multiple comparisons in statistical terms.

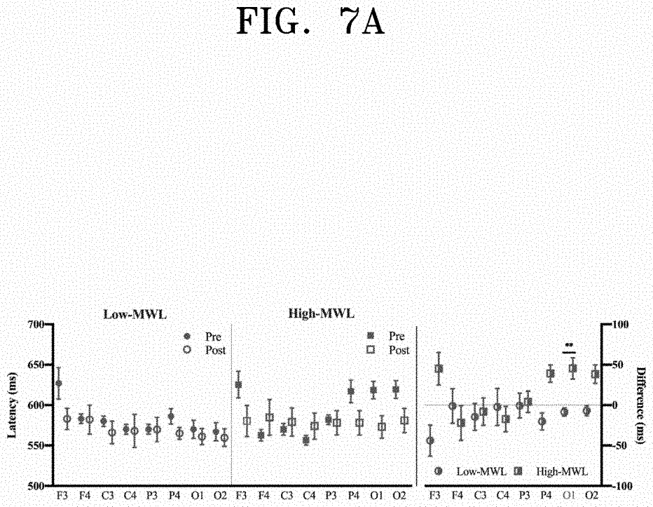

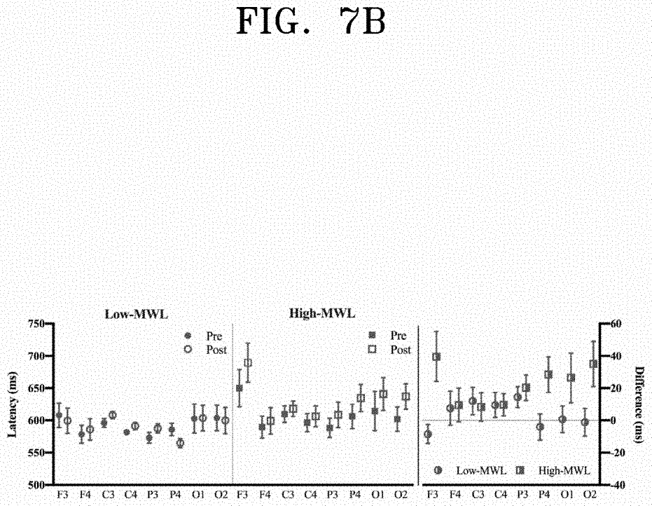

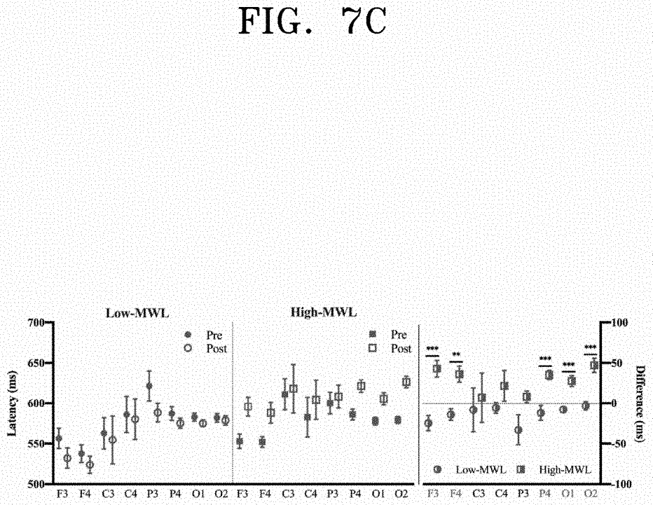

FIGS. 7A to 7C are statistical graphs illustrating, for respective channels, characteristics at 600 ms with respect to a time value (latency) of a peak. In detail, as the results of a paired sample T-test for a P600 latency time in an ERP between low-MWL and high-MWL conditions, FIG. 7A illustrates a statistical result in an ERP_{T} condition, FIG. 7B illustrates a statistical result in an ERP_{HEP} condition, and FIG. 7C illustrates statistical results in an ERP_{A-HEP} condition and the like (*p>0.05, **p>0.0031, ***p>0.001).

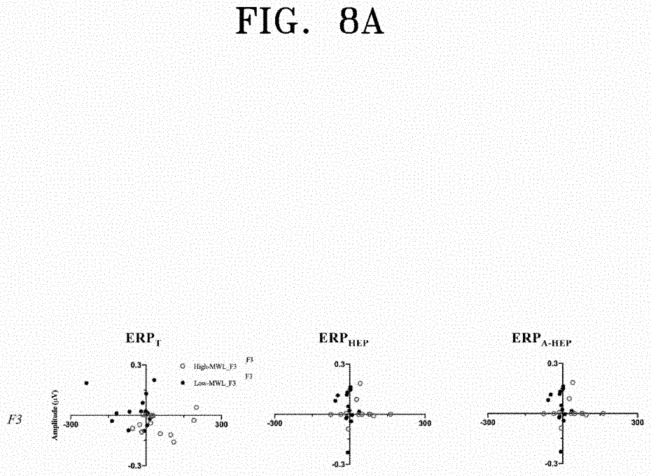

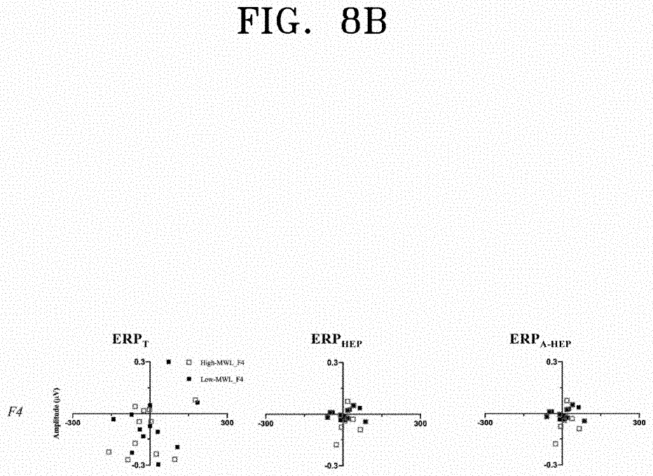

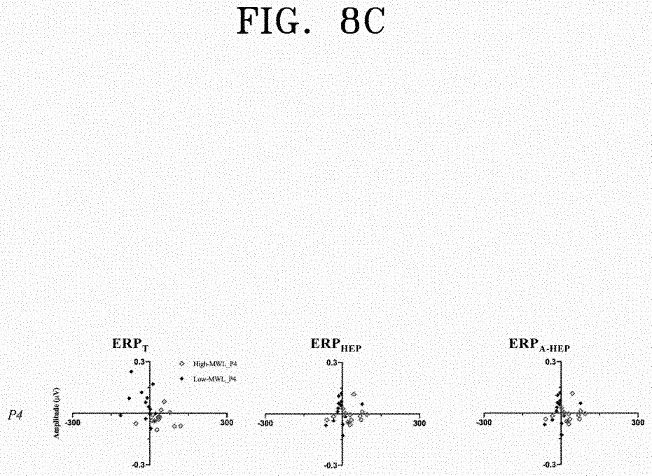

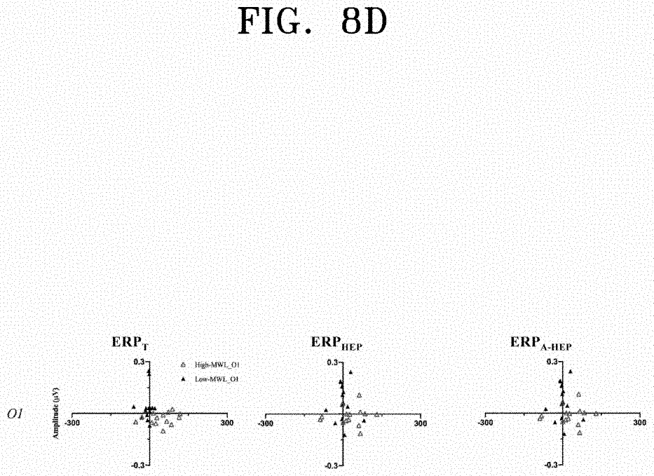

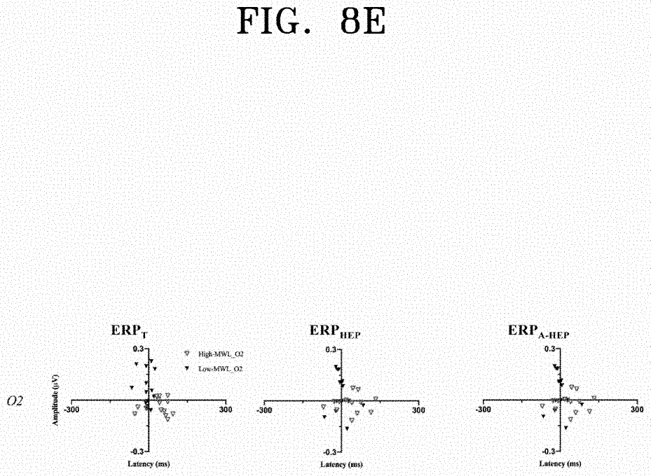

Also, FIGS. 8A to 8E illustrate graphs showing scatter plots for channels F3, F4, P4, O1, and O2 from among EEG channels. i.e., illustrate scatter plots of a high-MWL and a low-MWL in ERP_{T}, ERP_{HEP}, and ERP_{A-HEP} conditions plotted on orthogonal coordinates including latency (x-axis) and amplitude (y-axis).

As shown on the graphs, the classification statuses of two groups (the high-MWL and the low-MWL) may be directly observed by using amplitude and latency characteristics when comparing the two groups.

7. Result of Machine Learning Classification

A high-MWL and a low-MWL are classified in each condition by using RBF-SVM as a machine learning method. As shown in Table 3 below, the accuracy of 100%, which is significantly higher than in the other conditions, appears in the ERP_{A-HEP} condition.

TABLE 3

| | | Accuracy | Sensitivity | Specificity | |
|---|---|---|---|---|---|
| | Condition | (%) | (%) | (%) | AUC |
| RBF-SVM | ERP_{T} | 85.7 | 85.7 | 85.7 | 0.93 |
| (10-fold cross- | ERP_{HEP} | 71.4 | 64.3 | 78.6 | 0.76 |
| validation) | ERP_{A-HEP} | 100 | 100 | 100 | 1 |

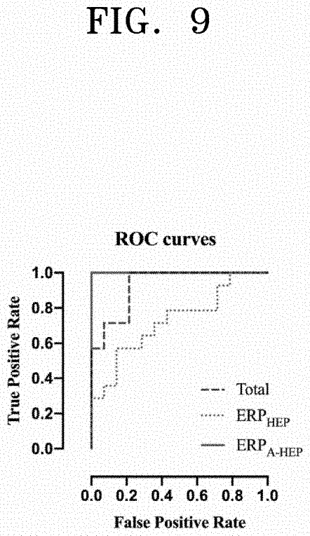

FIG. 9 illustrates receiver operation characteristic (ROC) curves with respect to ERP_{T}, ERP_{HEP} and ERP_{A-HEP} according to an RBF-SVM classifier.

8. ERP Stimulus Presentation System Considering HEP

According to the results of research, an ERP signal shows a difference when considering an HEP and when not considering the HEP. As a result, the difference appears differently in the results of statistics and machine learning classification. On the basis of the results of research, the present embodiment provides, as described below, a system that measures an ECG and reflects the ECG in real time to present stimulus such that characteristics to be obtained when presenting stimulus in an ERP experiment are not contaminated by an HEP.

The system provided herein uses a general-purpose QRS detection algorithm, and thus does not need a separate practice before an experiment. A user of the system follows stages below as in an existing ERP experiment.
1. Sensor Attachment: Electrodes for obtaining ECG and EEG signals are attached to the user. Lead-I type electrodes are attached for the ECG signal.
2. ERP Task: The user performs an ERP experiment while looking at ERP experiment stimulus presented on a screen.

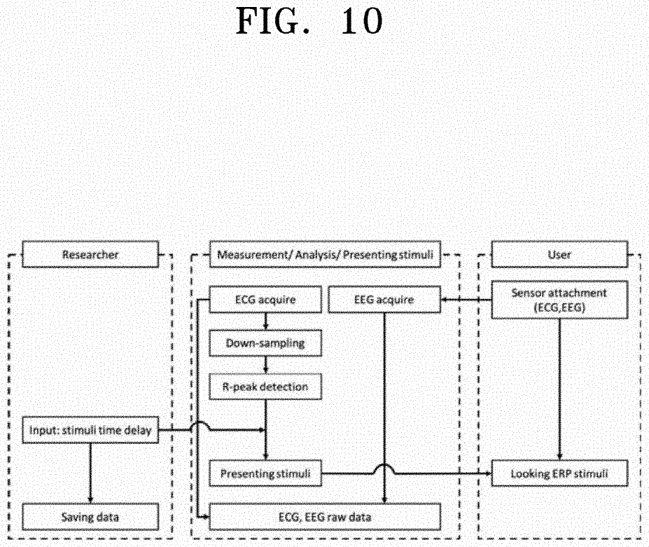

From the user's point of view, the ERP experiment is performed in the same method as in the existing experiment, but the ERP stimulus presentation system of the embodiment internally follows a process as described below, and FIG. 10 illustrates an algorithm of the entire system.
1. Input of ERP stimulus delay: The ERP stimulus presentation system determines whether to present ERP stimulus after how many ms after appearance (detection) of an R-peak according to the aim of the experiment. Here, an HEP, in particular, a first component of the HEP, which may affect during a period of about 50 ms to about 600 ms after the R-peak, needs to be considered.
2. Detection of R-peak of ECG: An R-peak is obtained in real time via a QRS detection algorithm.
3. Presentation of ERP stimulus: ERP stimulus is presented after preset delay on the basis of a point in time when the R-peak appears.
4. Repetitive Presentation of ERP stimulus: ERP stimulus is continuously presented on the basis of the R-peak being updated in real time.
5. Continuous Detection of ERP Signal: Detection of an EEG signal is continuously performed during the above process.

The present disclosure has a basic concept that ERP stimulus is not presented during a period of generation of an HEP signal affecting an EEG in a process of detecting an ERP signal.

A first component and a second component of the HEP signal occur after delay of a certain time after an ECG peak, and in ordinary, the HEP signal affects an EEG signal within about 250 ms to about 600 ms. The most effective method for denoising is not to present the ERP stimulus throughout a period of interference by the HEP signal. However, the non-presentation of the ERP stimulus over the entire period dramatically may reduce a stimulus exposure time for a subject and may therefore fail to detect a normal ERP signal. Therefore, according to another embodiment, even when the certain degree of intermixture of HEP noise is allowed, a partial section of the period of generation of the HEP signal may be applied.

According to an embodiment, ERP stimulus may not be presented during any one of a period of a first component of an HEP, in ordinary about 50 ms to about 250 ms, and a period of a second component, in ordinary about 250 ms to about 600 ms, or during a partial period within each section. Also, according to another embodiment, the ERP stimulus may not be presented during some period spanning both the first component (about 50 ms to about 250 ms) and the second component (about 250 ms to about 600 ms), e.g., during a period of about 200 ms to about 500 ms. In addition, according to another embodiment, a period for which the ERP stimulus is not presented may include only some time of the first component or the second component of the HEP signal.

According to one or more embodiments as described above, contamination of an ERP signal by an HEP may be prevented or reduced, thereby improving the accuracy of classification of the ERP signal.

According to one or more embodiments, an effect of the HEP induced by conduction may be excluded, or an attenuated ERP signal may be detected. In an embodiment, an ERP signal in which an effect of an ECG as continuously occurring noise is excluded may be obtained by presenting ERP stimulus as a cognitive load to a subject after a certain period of time has elapsed based on a time point of the occurrence of the ECG. The ERP signal obtained as described above is not affected by the ECG, and thus, patterns of ERP components (N2, P3, N4, P6, and the like) for an event, such as sensory or cognitive stimulus or movement in the brain, clearly appear compared to those affected by an HEP, and classification for the event may be more accurately performed.

Example embodiments have been described and shown in the accompanying drawings to help the understanding of the present disclosure. However, it should be understood that these embodiments are merely illustrative of the present disclosure and do not limit the present disclosure. Also, it should be understood that the present disclosure is not limited to what has been shown and described. Therefore, various other modifications may be made by one of ordinary skill in the art.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method of detecting an event-related potential (ERP) signal, the method comprising:
detecting an R-peak signal by detecting an electrocardiogram (ECG) signal of a subject by an ECG sensor;
inducing an evoked potential to the subject by presenting ERP stimulus to the subject for a certain period on the basis of the R-peak signal; and
detecting an electroencephalogram (EEG) signal of the subject exposed to the ERP stimulus by using an EEG sensor and extracting an ERP signal from the EEG signal, wherein, after a latency by a certain time from a point in time when the R-peak occurs, the ERP stimulus presented to the subject during the certain period is removed to inhibit intermixture of a heart-rate evoked potential (HEP) of the subject with the ERP signal.

2. The method of claim 1, wherein the certain period includes a time range corresponding to at least one of a first component and a second component of the HEP.

3. The method of claim 1, wherein the ERP stimulus is presented to the subject after about 50 ms to about 600 ms from the point in time when the R-peak occurs.

4. The method of claim 1, wherein the HEP includes a first component and a second component, and the ERP stimulus is presented to the subject from a time point beyond a point in time when the first component of the HEP occurs.

5. The method of claim 1, wherein the HEP is detected from the subject, and the certain period for which the ERP stimulus is not presented is calculated from the HEP obtained from the subject.

6. An apparatus for detecting an event-related potential (ERP), the apparatus comprising:
a simulator configured to induce an evoked potential to a subject by presenting an ERP stimulus to the subject;
an electrocardiogram (ECG) measurer having an ECG sensor configured to detect an ECG signal from the subject exposed to the ERP stimulus;
an electroencephalogram (EEG) measurer having an EEG sensor configured to detect an EEG signal from the subject;
a signal processing unit configured to extract a heart-rate evoked potential (HEP) signal by detecting an R-peak from the ECG signal, and detect an ERP signal from the EEG signal; and
an ERP stimulus controller configured to inhibit intermixture of an HEP of the subject with the ERP signal by removing the ERP stimulus presented to the subject during a certain period, after a latency by a certain time from a time point of occurrence of the R-peak detected from the ECG signal by the signal processing unit.

7. The apparatus of claim 6, wherein the certain period includes a time range corresponding to at least one of a first component and a second component of the HEP.

8. The apparatus of claim 6, wherein the ERP stimulus is presented to the subject after about 50 ms to about 600 ms from the time point of the occurrence of the R-peak.

9. The apparatus of claim 6, wherein the HEP includes a first component and a second component, and the ERP stimulus is presented to the subject from a time point beyond a point in time when the first component of the HEP occurs.

10. The apparatus of claim 6, wherein the HEP is detected from the subject, and the certain period for which the ERP stimulus is not presented is calculated from the HEP obtained from the subject.
